(19) United States
(12) Patent Application Publication
(10) Pub. No.: US 2012/0094359 A1
(43) Pub. Date: Apr. 19, 2012

(54) Novel System and Method of Anaerobic Fermentation

(75) Inventors: Michael Torres, Irvine, CA (US); Josh McElfresh, Orange, CA (US); Tsu-Shun Lee, Irvine, CA (US)

(73) Assignee: ALLERGAN, INC., Irvine, CA (US)

(21) Appl. No.: 13/273,052

(22) Filed: Oct. 13, 2011

Related U.S. Application Data
(60) Provisional application No. 61/392,791, filed on Oct. 13, 2010.

(51) Int. Cl. C12N 1/20

(52) U.S. Cl. 435/252.1

(57) ABSTRACT

The present invention relates to growing anaerobic bacteria by measuring the reduction potential of a growth media at a start time and determining a grow time by correlating the start time reduction potential with said reduction coefficient to calculate when said second anaerobic fermentation system will reach a sufficiently low oxygen concentration to enable growth for an anaerobic bacteria, then adding the anaerobic bacteria to the growth media at a time no sooner than the grow time.

This application claims priority to U.S. Provisional Application No. 61/392,791, filed Oct. 13, 2010, entirely incorporated by reference.

### FIELD OF THE INVENTION

The present invention relates to improved methods for growing anaerobic bacteria in fermentation systems.

### BACKGROUND

Fermentation of anaerobic bacteria have been known for decades. See, e.g., U.S. Pat. No. 2,348,448 to Brewer, U.S. Pat. No. 4,476,224 to Adler and U.S. Pat. No. 5,955,344 to Copeland et al., each incorporated entirely by reference. Anaerobic fermentation of obligate anaerobes requires suitable starting media. Therefore, prior to inoculation, the media is maintained in a sterile, oxygen-free environment to allow the dissolved oxygen to reach a near steady-state zero level. To facilitate this reduction in dissolved oxygen, the media is exposed to a nitrogen (N₂)-rich, oxygen-deficient environment; this facilitates the leaching of dissolved oxygen from the media to the overhead space.

The rate at which fermentation media reaches an anaerobic state is impacted by many variables, including system design, engineering control parameters, and numerous process inputs. Though widely used, the empirical approach to media characterization is very inefficient, requiring the initiation and maintenance of multiple bacterial cultures as well as frequent testing in order to evaluate the effectiveness of the reduction process used.

### BRIEF DESCRIPTION OF THE FIGURES

FIG. 1 is a plot of relative reduction potential vs. time for a small scale run with a first set of conditions.

FIG. 2 is a plot of relative reduction potential vs. time for a small scale run with a second set of conditions.

FIG. 3 is a plot of relative reduction potential vs. time for a large scale run with a first set of conditions. FIG. 3a plots the relative reduction potential vs. time, and FIG. 3b plots the log of relative reduction potential vs. time.

FIG. 4 is a diagram of relative reduction potential vs. time for a large scale run with a second set of conditions. FIG. 4a plots the relative reduction potential vs. time, and FIG. 4b plots the log of relative reduction potential vs. time.

### DESCRIPTION

The present invention relates to methods for characterizing a media culture. In particular, the present invention relates to the use of mass-transfer theory to accurately determine media conditions.

A highly efficient fundamental approach based on mass transfer theory is presented here. A mass transfer model was built and validated in order to describe the reduction process. A series of experiments were performed at small (spinner flask) and large (fermentor) scales in order to demonstrate the model's accuracy and capabilities. The anaerobic fermentation reduction process was successfully characterized by using this fundamental approach.

The present invention requires few resources, and can be tested using only medium without the necessity for anaerobic culturing. The predictive model can be utilized for process technology transfer, scale-up, and scale-down at different sites and laboratories. This concept of applying first-principle science to process development can eliminate the trial and error approach, thus saving resources, time and expense.

Model Derivation.

The following equations were utilized to determine media oxygen levels:

$$O T R = \frac{d {C}_{L}}{d t} = {k}_{R} ( {C}_{L}^{*} - {C}_{L} ) ( \frac{A}{V} ) \qquad Equation 1$$

where, k_{R} is the reduction coefficient, A is the total gas-liquid contact surface area, V is the total liquid volume, C_{L} is the oxygen concentration of the liquid, and C*_{L} is the saturation dissolved oxygen concentration. The A/V term can be replaced with a and combined with the k_{R} term to yield an alternative form of the reduction coefficient, k_{R}a. Equation 1 can be rearranged to yield Equation 2:

$$\frac{d {C}_{L}}{d t} = {k}_{R} a ( {C}_{L}^{*} - {C}_{L} ) \qquad Equation 2$$

Integration of Equation 2 yields Equation 3 when C_{L} equals zero at time zero:

$$ln ( \frac{{C}_{L}^{*}}{{C}_{L}^{*} - {C}_{L}} ) = {k}_{R} a ( t ) \qquad Equation 3$$

Assuming ORP_{L}∝C_{L} and substituting absolute ORP values for dissolved oxygen in Equation 3 yields Equation 4:

$$ln ( \frac{O R {P}_{L}^{*}}{O R {P}_{L}^{*} - O R {P}_{L}} ) = {k}_{R} a ( t ) \qquad Equation 4$$

where, ORP_{L} is the value at time of measurement, and ORP*_{L} is the lowest measured ORP_{L} value for each run.

Equation 4 was modeled using the formula y=mx+b, where k_{R}a corresponds to the slope, m, and c is the y-intercept, b. A linear regression of medium ORP (oxidation-reduction potential) profiles yielded a k_{R}a for each set of experimental conditions. These k_{R}a values for each run can be compared statistically across a study.

Changes to current processes may be implemented based on positive impact to k_{R}a response; the faster rate of oxidation-reduction potential decrease (a proxy for dissolved oxygen reduction) from the fermentation medium decreased total process time. Parameter ranges were proven acceptable based on statistical impact to k_{R}a response. Additionally, Min and Max (k_{R}a response) studies were performed to identify process capabilities.

The simple predictive model (linear equation) built using the experimental data predicts the total time to dissolved oxygen steady-state zero levels is provided in Equation 5:

Time=A+B(Filter Surface Area) Equation 5:

The predictive power of this model was tested against known control process parameters. Further, an additional experiment was performed to gauge model efficiency. The model accurately predicted the total time required to achieve appropriate oxygen levels, within ±5%(95% accuracy). The present invention may be used during technical transfer from one facility to another, to evaluate prospective changes to filter surface area, as well as a scale-up/scale-down guide, etc.

### EXAMPLES

Example 1

A series of small scale and large scale DOE (design of experiments) based studies were performed; parameters and tested ranges were chosen based on potential impact to rate of dissolved oxygen reduction. Independent variables which may be tested include but are not limited to:
a) Gas rate (overlay and sparge);
b) Surface to volume ratio;
c) System agitation rate;
d) System temperature;
e) Vessel geometry;
f) Vessel pressure;
g) Impeller type and position; and,
h) Medium composition (viscosity, etc.).

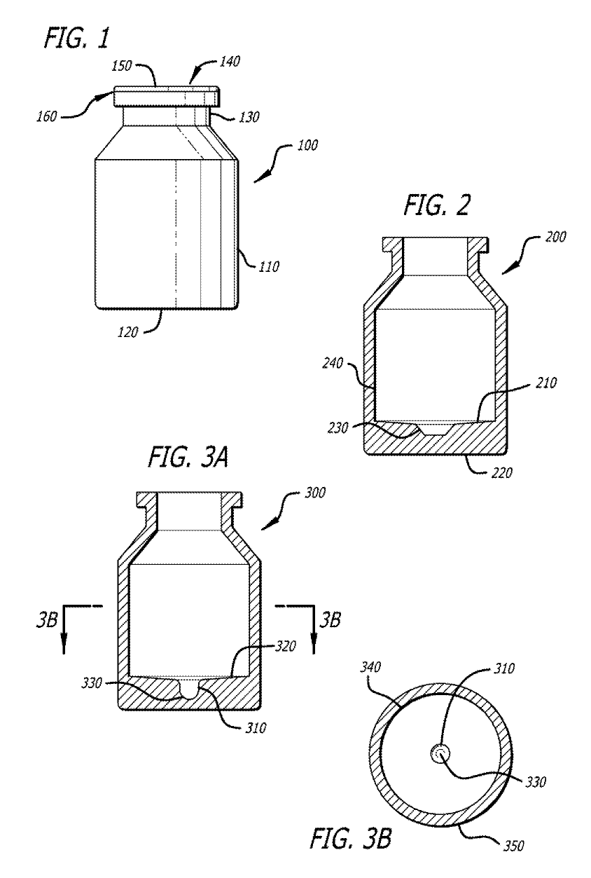

Since the redox probe was standardized before every run, the values may be offset by as much as ±20 mV. Additionally, once medium and probe were sterilized, the probe was used without restandardization. It is therefore more appropriate to observe the relative change in ORP rather than the absolute ORP_{L} value. The relative change in ORP was calculated by taking the absolute value of ORP*_{L} minus ORP_{L} at each time point; the minimum measured ORP_{L} value for each experiment was assumed to be the steady-state value (ORP*L) and used in the calculations. The relative change in ORP was then plotted versus time (minutes) as shown in Figures 1a, 2a, 3a and 4a. The relative change in ORP (ORP*_{L} minus ORP_{L}) versus time data was inserted into Equation 4 and plotted versus time (as shown in Figures 1b, 2b, 3b and 4b) to determine the k_{R}a value (the slope of the linear regression line) for each set of experimental conditions. Applicants note that absolute ORP_{L} values may also be used to determine k_{R}a.

### Example 2

Bottles of growth media were prepared for reduction and placed in an anaerobic chamber. Each bottle had a different size vent filter, creating a difference in the rate which oxygen could diffuse from each bottle. The ORP for each bottle was measured over the course of the reduction period. The data from the reduction period was transformed using Equation 4 to calculate k_{R}a. FIGS. 1 and 2 show the results for the vent filter surface areas 7.5 cm² and 19.6 cm², respectively. The bottle with 7.5 cm² vent filter had a k_{R}a of 0.0014 and the bottle using a 19.6 cm² vent filter had a k_{R}a of 0.0021. This result confirmed that reduction rate could be predicted calculating k_{R}a.

### Example 3

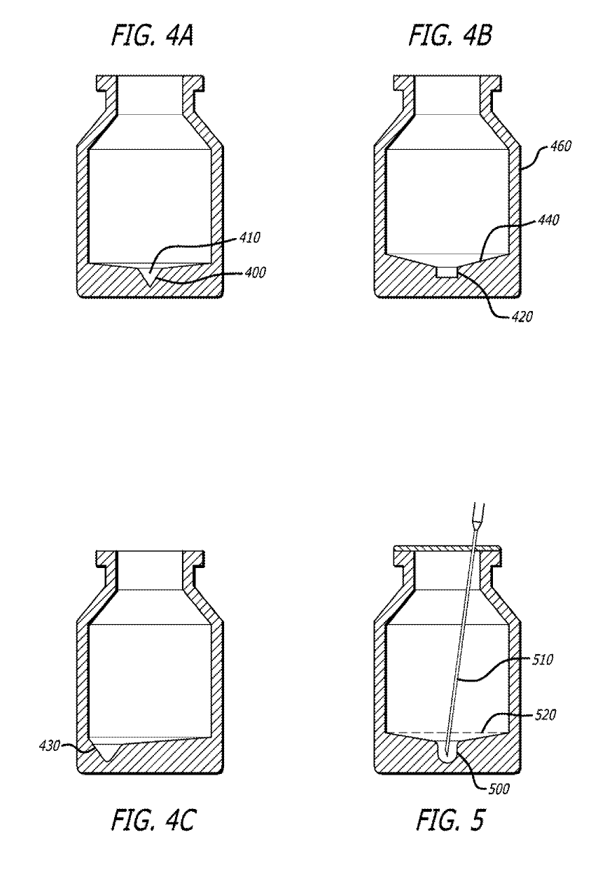

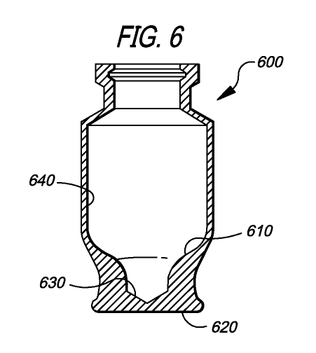

Fermentors were filled with growth media and nitrogen was pumped through the headspace of these vessels. The rate at which nitrogen was circulated was varied to create a difference in reduction rate. The ORP for each fermentor was measured over the course of the reduction period. The data from the reduction period was transformed using Equation 4 to calculate k_{R}a. FIGS. 3 and 4 show the results for the lowest and highest nitrogen flow rates, respectively. The fermentor with a nitrogen flow rate of 4 slpm showed a k_{R}a of 0.003 and the fermentor with a nitrogen flow rate of 20 slpm showed a k_{R}a of 0.004. This duplicated the result seen at the small scale, again showing that k_{R}a predicts reduction rate.

### Example 4

A second fermentation was performed with the fermentation system described in Example 3, with k_{R}a=0.004. The calculated grow time was nine hours. After the grow time, the media was inoculated. Optical density was measured throughout the duration of the fermentation, peaking at 9.405 OD540. This successful run confirmed that the time estimates obtained using calculated k_{R}a values were useful.

We claim:

1. A method of growing anaerobic bacteria comprising:
a) setting up an anaerobic fermentation system comprising growth media with a set of a first system conditions;
i) measuring the reduction potential in said growth media over a plurality of time points;
ii) calculating a reduction coefficient for said set of a first system conditions;
b) setting up a second anaerobic fermentation system comprising growth media with said set of a first system conditions;
i) measuring the reduction potential of said growth media at a start time;
ii) determining a grow time by correlating the start time reduction potential with said reduction coefficient to calculate when said second anaerobic fermentation system will reach a sufficiently low oxygen concentration to enable growth for an anaerobic bacteria;
c) adding said anaerobic bacteria to said growth media in said second anaerobic fermentation system no sooner than said grow time;
d) growing said anaerobic bacteria in said second anaerobic fermentation system.

2. The method of claim 1, wherein the reduction coefficient is calculated using Equation 4.

3. The method of claim 1, wherein the anaerobic bacterial growth is part of a technical transfer from a first facility to a second facility.

4. The method of claim 1, wherein the anaerobic bacterial growth is part of a scale-up process.
